(19) United States
(12) Patent Application Publication
(10) Pub. No.: US 2025/0255665 A1
(43) Pub. Date: Aug. 14, 2025

(54) METHODS FOR TREATMENT OF NASAL WALL INSUFFICIENCY, AS WELL AS PUNCTURE GUIDE, INSTRUMENT SET AND MEDICAL SYSTEMS THERFORE

(71) Applicant: OLYMPUS WINTER & IBE GMBH, Hamburg (DE)

(72) Inventors: Thomas STEIN, Teltow (DE); Eva HERZOG, Berlin (DE); Katica SCHÜRSTEDT, Schönefeld (DE); Wladislaw WECHSLER, Hamburg (DE); Etienne Michael STOCKHAUSEN, Dallgow-Döberitz (DE); Matteo LAZZARI, Hamburg (DE)

(73) Assignee: OLYMPUS WINTER & IBE GMBH, Hamburg (DE)

(21) Appl. No.: 19/049,885

(22) Filed: Feb. 10, 2025

Related U.S. Application Data
(60) Provisional application No. 63/551,391, filed on Feb. 8, 2024.

(51) Int. Cl. A61B 18/14; A61B 18/00

(52) U.S. Cl. CPC A61B 18/1477; A61B 2018/00327; A61B 2018/1425

(57) ABSTRACT

Provided herein is a method for treatment of nasal wall insufficiency, comprising: providing a puncture guide, the puncture guide comprising a stabilizing frame and a plurality of puncture passages; providing a bipolar radiofrequency probe having a piercing tip; introducing the puncture guide into the nasal passages of the patient; piercing the radiofrequency probe into nasal wall tissue through one of the plurality of puncture passages; energizing the radiofrequency probe for heating the nasal wall tissue; de-energizing the radiofrequency probe; removing the radiofrequency probe from the nasal wall tissue; repeating the above steps for sequentially pierce the radiofrequency probe through each of the plurality of puncture passages; and allowing the nasal wall tissue to cool.

Further provided are a puncture guide for treatment of nasal wall insufficiency, comprising a stabilizing frame and a plurality of puncture passages; an instrument set for treatment of nasal wall insufficiency, comprising: a bipolar radiofrequency probe, the probe having a main body and an elongate shaft, the shaft having a piercing tip and first and second electrodes disposed near the distal end thereof, and a puncture guide; and a medical system for treatment of nasal wall insufficiency, the system comprising: a bipolar radiofrequency probe, the probe having a main body and an elongate shaft, the shaft having a piercing tip and first and second electrodes disposed near the distal end thereof; a puncture guide having a stabilizing frame and a plurality of puncture passages; and an electrosurgical generator.

The present application claims benefit of the U.S. provisional application 63/551,391 filed on Feb. 8, 2024.

### FIELD

The present disclosure relates to methods, puncture guides, instrument sets, and systems for treatment of nasal wall insufficiency. More particularly, the present disclosure relates to electrosurgical methods for treatment using a puncture guide, puncture guides, and instrument sets and systems including a puncture guide.

### BACKGROUND

Nasal wall insufficiency is a common cause of obstructed nasal breathing in adult patients. Patients suffering from nasal wall insufficiency experience difficulties inhaling a sufficient volume of air when inhaling through the nose, caused by a dynamic collapse of the lateral nasal walls due to limited strength of cartilage tissue around the nasal passage. Classical treatments for nasal wall insufficiency include grafting, implants, and suture suspension.

Recently, electrosurgical therapy options have been proposed, wherein lesions are created in the affected tissue with the aim to increase the stiffness thereof. However, the proposed methods are complicated, and still involve surgery.

It would be attractive to provide methods, instrument sets, and systems for easy and effective treatment of nasal wall insufficiency.

### SUMMARY

The present disclosure provides a method for treatment of nasal wall insufficiency, comprising: providing a puncture guide, the puncture guide comprising a stabilizing frame and a plurality of puncture passages; providing a bipolar radiofrequency probe having a piercing tip; introducing the puncture guide into the nasal passages of the patient; piercing the radiofrequency probe into nasal wall tissue through one of the plurality of puncture passages; energizing the radiofrequency probe for heating the nasal wall tissue; de-energizing the radiofrequency probe; removing the radiofrequency probe from the nasal wall tissue; repeating the previous steps for sequentially pierce the radiofrequency probe through each of the plurality of puncture passages; and allowing the nasal wall tissue to cool.

The present inventors have discovered that electrosurgical heating of the nasal wall tissue while the tissue is stabilized by a frame of the puncture guide can achieve a stiffening effect on the nasal wall tissue, and can improve the strength of the nasal wall tissue to maintain a shape provided by the stabilizing frame of the puncture guide.

The puncture guide may be left in the nasal passages of the patient until the method is completed. In some embodiments, the puncture guide is removed from the nasal passage of the patient about 2 minutes after de-energizing the radiofrequency probe for the last time. The nasal wall tissue may conform to a shape of the puncture guide during treatment, and may fully or partially maintain the amended shape after cooling.

In some embodiments, the puncture guide may be configured to be introduced into both nasal passages of the patient simultaneously. In other embodiments, the puncture guide may be configured to be introduced into the nasal passages of the patient one after the other.

Further provided herein is a puncture guide for treatment of nasal wall insufficiency, the puncture guide having a stabilizing frame and a plurality of puncture passages. The instrument set may be configured to allow application of the methods described above.

The frame of the puncture guide may include at least one hollow plug. The frame of the puncture guide may include a first hollow plug for introduction into the right nasal passage of a patient, and a second hollow plug for introduction into the left nasal passage of a patient. The frame of the puncture guide may include a tether between the first hollow plug and the second hollow plug.

The first hollow plug may comprise a first plurality of puncture passages. The second hollow plug may comprise a second plurality of puncture passages. The puncture guide may include a handle attached to the hollow plug or hollow plugs.

In some embodiments, the puncture guide may comprise a first guide plate and a first arm extending proximally from the first guide plate, a second guide plate and a second arm extending proximally from the second guide plate, and a plurality of puncture passages provided in the first guide plate. The puncture guide may further comprise a plurality of puncture passages provided in the second guide plate. The first and the second guide plates may have a curved shape. The first arm and the second arm may merge in a proximal elastic joint.

Also provided herein is an instrument set for treatment of nasal wall insufficiency, the instrument set comprising a bipolar radiofrequency probe, the probe having a main body and an elongate shaft, the shaft having a piercing tip and first and second electrodes disposed near the distal end thereof, and a puncture guide as described herein.

The present disclosure further provides a medical system for treatment of nasal wall insufficiency, the system comprising: a bipolar radiofrequency probe, the probe having a main body and an elongate shaft, the shaft having a piercing tip and first and second electrodes disposed near the distal end thereof, a puncture guide having a stabilizing frame and a plurality of puncture passages, and an electrosurgical generator.

### BRIEF DESCRIPTION OF THE DRAWINGS

The subject of the disclosure is in the following further described at hand of exemplary drawings. The drawings, which are not necessarily drawn to scale, are provided for better understanding of the concepts disclosed herein, without the intention to limit the scope of disclosure.

In the drawings:

FIG. 1 shows steps of a method according to the present disclosure,

FIG. 2 shows a medical system according to the present disclosure,

FIG. 3 shows a bipolar radiofrequency probe,

FIGS. 4a-4c show a puncture guide,

FIGS. 5a-5c show a further puncture guide.

### DETAILED DESCRIPTION

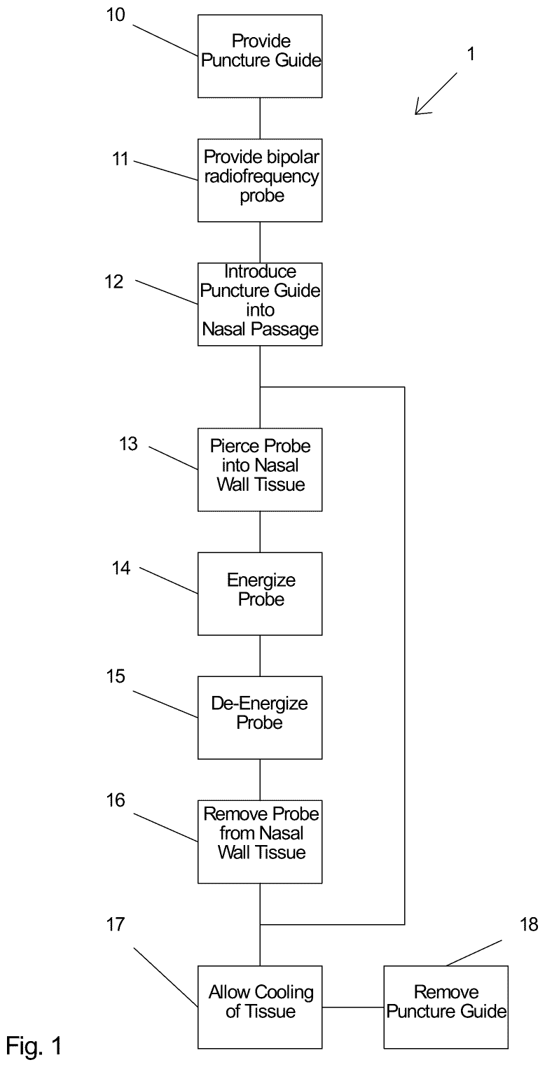

FIG. 1 shows an exemplary method 1 for treatment of nasal wall insufficiency. It will be appreciated that only the relevant steps of the procedure are explained in detail. Practitioners may add intermediate routine steps like administration of local anesthesia.

In step 10, a puncture guide is provided. The puncture guide, examples of which will be described in more detail herein below, comprises a stabilizing frame and a plurality of puncture passages. The stabilizing frame may be shaped to define a desired geometry of a patient's nasal passage when the puncture guide is inserted into the patient's nasal passage.

In step 11, a bipolar radiofrequency probe is provided. The radiofrequency probe may comprise a main body, serving as a handle, and an elongate shaft. The shaft of the radiofrequency may comprise a piercing tip. The radiofrequency probe may comprise first and second electrodes at a distal end thereof.

In step 12, the puncture guide is introduced into the nasal passage of the patient. Introducing the puncture plug into the nasal passage of the patient may elastically push nasal wall tissue of the patient into a desired shape.

In step 13, the radiofrequency probe is pierced through one of the puncture passages of the puncture guide into the nasal wall tissue. The radiofrequency probe may be inserted into the nasal wall tissue deep enough so that both first and second electrodes of the radiofrequency probes are completely buried in nasal tissue.

In step 14, the radiofrequency probe is energized to heat the nasal wall tissue. For energizing, the radiofrequency probe is connected to an electrosurgical generator, and the electrosurgical generator is activated to provide electrosurgical energy to the radiofrequency probe. Heating the tissue can create a tissue lesion in the nasal wall tissue, which increases stiffness of the tissue.

After a desired lesion size has been created, the radiofrequency probe is de-energized in step 15. Desirable lesion sizes may range between 5 mm and 15 mm in length, and 1.5 mm to 2.5 mm in width. A desired lesion size may be 11 mm in length and 2 mm in width. For de-energizing the radiofrequency probe, the electrosurgical generator may be deactivated.

Then, in step 16, the radiofrequency prove is removed from the nasal wall tissue by pulling the shaft of the probe through the puncture passage of the puncture guide.

The method then loops back to step 13, where the radiofrequency probe is pierced through a next puncture passage of the puncture guide. Then, the probe is again energized to create another lesion close to the previously created lesion, and de-energized and removed.

The same may be repeated until the radiofrequency probe has been pierced through all puncture passages of the puncture guide. In this way, a predetermined number of lesions may be created in the nasal wall tissue of the patient. The predetermined number of lesions may be two lesions, three lesions, four lesions, of even more than four lesions.

After the radiofrequency probe has been pierced through all puncture passages of the puncture guide, in step 17 the radiofrequency probe is removed from the site, while the puncture guide is left in the nasal passage for a predetermined time span, while the nasal wall tissue of the patient is allowed to cool. When the nasal wall tissue of the patient cools down, cartilage in the nasal wall further stiffens, while maintaining a shape defined by the stabilizing frame of the puncture guide. Cooling of the nasal wall tissue may be assisted by external or internal application of a cooled fluid like air or water, or by external application of a cooling pad. The time span for cooling of the nasal wall tissue may be between 1 minute and 5 minutes. The time span for cooling of the nasal wall tissue may be about 2 minutes.

Finally, the puncture guide is removed from the nasal passage of the patient in step 18.

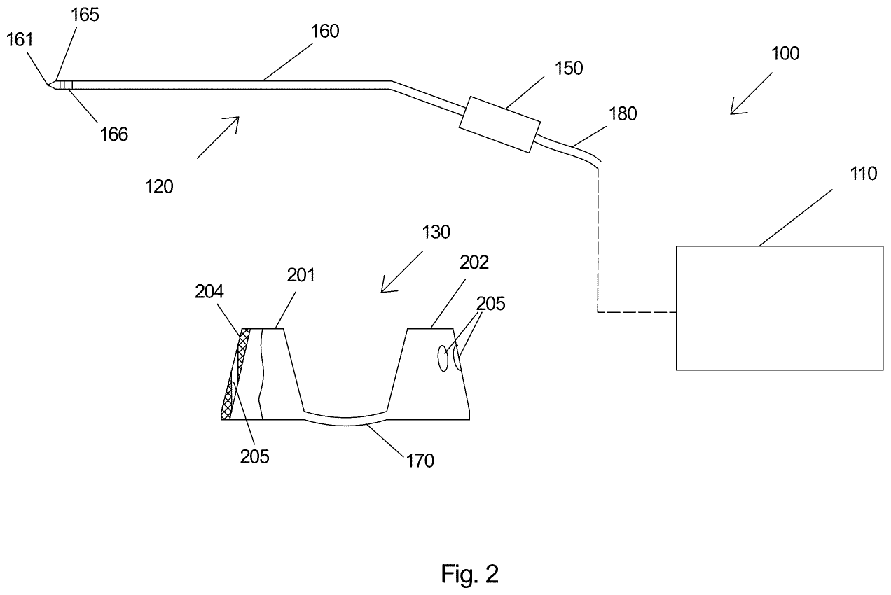

FIG. 2 shows a medical system 100, which can be used to perform a method as described above. The system 100 includes an electrosurgical generator 110, a bipolar radiofrequency probe 120, and a puncture guide 130.

The electrosurgical generator 110 may be any suitable electrosurgical generator like from the ESG series offered by the present applicant. It is configured to provide the bipolar radiofrequency probe 120 with an electrosurgical therapy signal.

The bipolar radiofrequency probe 120 comprises a main body 150 and a rigid elongate shaft 160 extending distally therefrom. At the distal end, the shaft 160 comprises a piercing 161 for facilitating insertion of the shaft 160 into nasal wall tissue (not shown). In some embodiments, the shaft 160 may have a diameter of about 1 mm, and may have a length of about 40 mm to 100 mm, preferably of about 50mm to 60 mm.

Near the distal end, the shaft 160 comprises two electrodes 165, 166, which contact tissue (not shown) to couple the electrosurgical therapy signal into the tissue. In the shown embodiment, one of the electrodes 165 forms the piercing tip 161.

The main body 150 is sized to enable easy and convenient handling of the radiofrequency probe 120. Conductors (not shown) from the electrodes 165, 166 run through the shaft 150 and are connected to conductors of a supply cable 180 inside of the main body.

The puncture guide 130 shown in FIG. 2 comprises two frustoconical plugs 201, 202 linked by a tether 203. The plugs 201, 202 each have a hollow passage defined by a wall 204, allowing a patient to breath normally when the puncture guide 130 is inserted into the patient's nose.

In the wall 204 of the plugs 201, 202, puncture passages 205 are provided. As can be seen from the cutaway section of the left plug 201, the puncture passages run about parallel to a longitudinal axis of the plugs 201, 202, so that the radiofrequency probe 120 can be pierces through the puncture passage 205 into the nasal wall (not shown) of the patient in a shallow angle, without piercing though the nasal wall tissue. The term “about parallel” as used herein may encompass a small angular deviation between the puncture passages 205 and the longitudinal axis of the plugs 201, 202, which may be in the range of ±1°, ±2°, ±5°, or the like.

In the shown embodiment, each plug 201, 202 comprises three puncture passages 205, of which only on is visible in the breakout section of plug 201, and two are visible on the outer surface of plug 202. In other embodiments, puncture passages may be provided in a different number like two puncture passages per plug, four puncture passages per plug, or even more puncture passages per plug.

The wall 204 of the plugs 201, 202 forms a stabilizing frame of the puncture guide 120. The material of the wall 204 is selected to be sufficiently biocompatible so that the puncture guide 120 may safely be placed in the nose of a patient for a period ranging from a few minutes to about one hour. The material of the wall 204 may further be selected to provide sufficient stiffness so that nasal wall tissue of the patient may be pushed into and maintained in a desired shape during treatment. Applicable materials include surgical steel, biocompatible polymers, or the like. In case of polymers, the plugs 201, 202 and the tether 203 may be integrally formed as one piece. In other embodiments, the plugs 201, 202 and the tether 203 may be formed as separate parts and may be connected through any suitable means.

The bipolar radiofrequency probe 120 and the puncture guide 103 of the shown embodiment are designed as single-use instruments. The bipolar radiofrequency probe 120 and the puncture guide 130 may be provided as pre-confectioned instrument set, which is packed ready to use. A pre-confectioned instrument set may comprise a single radiofrequency probe and a single puncture guide. In some embodiments, a pre-confectioned instrument set may comprise a plurality of puncture guides, which may have different sizes to fit with different patient anatomies. In other embodiments, a pre-confectioned instrument set may comprise a plurality of radiofrequency probes having different shaft lengths, different shaft diameters, or both.

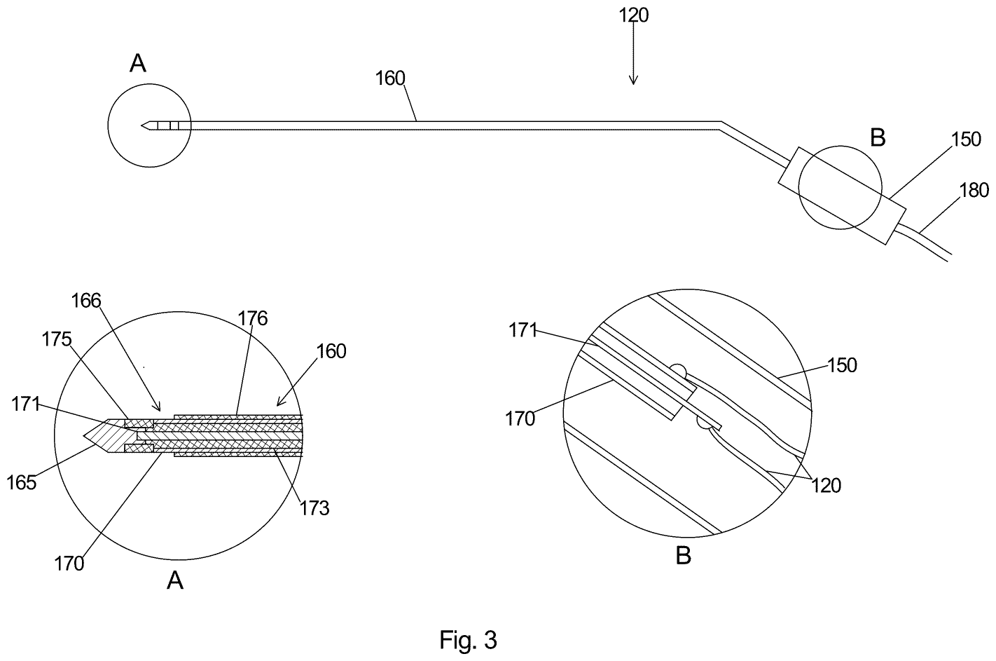

FIG. 3 shows the bipolar radiofrequency probe 120 in more detail. The probe 120 comprises a main body 150 with a rigid elongate shaft 160 extending distally therefrom. The shaft 160 is bent near the main body 150 to facilitate introduction of the probe into the puncture guide 130.

At a distal end of the shaft 160, the probe 120 comprises a pair of electrodes 165, 165 spaced apart from each other along the longitudinal axis of the shaft 160. The distal electrode 165 has a pointed tip suitable for piercing into tissue.

As can be seen from detail A of FIG. 3, the shaft 160 of the probe 120 includes a conductive shaft tube 170, which can be made from surgical steel or any other suitable metallic material. Within the conductive tube 170, a center wire 171 runs along the shaft 160. An inner insulator 173 isolates the center wire from the conductive tube. The conductive tube 170 is sufficiently rigid to avoid bending of the shaft 160.

At the distal end of the shaft 160, the center wire 171 projects from the conductive tube 170 and is connected to the distal electrode 165 by any suitable means like soldering or crimping. An insulating washer 175 isolates the distal electrode 165 from the conductive tube 170.

An insulating sheath 176 covers a main part of the outside of the conductive tube 170, leaving only a short distal portion uncovered. The uncovered portion of the conductive tube forms the proximal electrode 166.

As shown in detail B of FIG. 3, in the main body 150, the conductive tube 170 and the center wire 171 are connected to respective wires of a supply cable 180, which can be connected to a radiofrequency generator through a plug (generator and plug not shown in FIG. 3). The main body 150 can be made from any suitable material like thermoplastic resin, and can be filled with potting (not shown) to secure the other components of the probe.

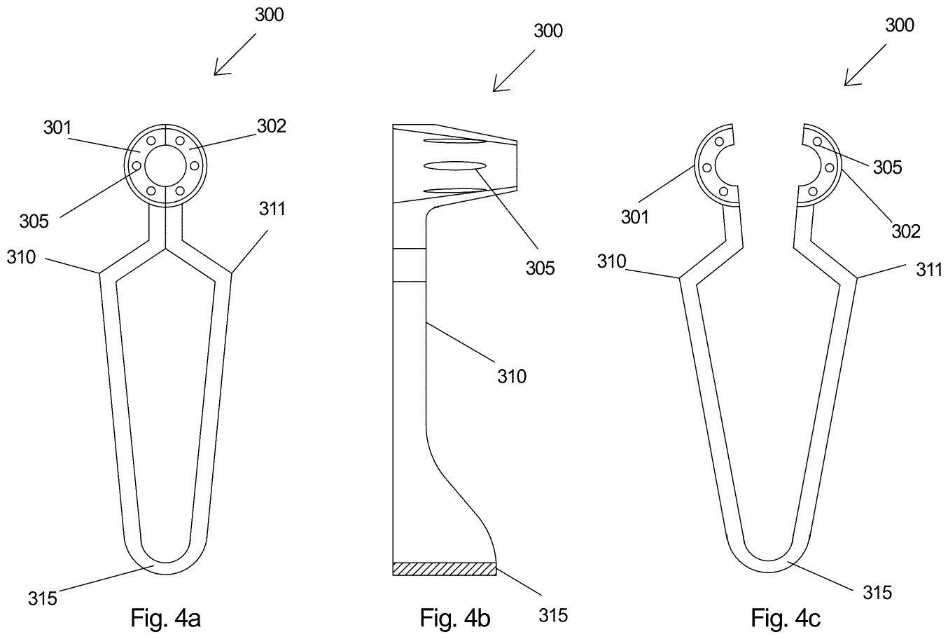

FIGS. 4a to 4c show a further embodiment of a puncture guide 300 to be part of an instrument set instead of puncture guide 120.

Puncture guide 300 comprises two curved guide plates 301, 302, which can be moved between a closed position, shown in FIG. 4a, and an expanded position shown in FIG. 4c. FIG. 4b shows the puncture guide 300 in a sectional side view.

In the closed position, the guide plates 301, 302 are brought into contact to form a plug similar to the plugs 201, 202 shown in FIG. 2. In the shown embodiment, each guide plate 301, 302 comprises three puncture passages 305. In the expanded position, the guide plates 301, 302 are separated from each other.

Arms 310, 311 are attached to the guide plates, and are connected by an elastic joint 315 at their other ends. The elastic joint 315 is configured to bias the guide plates 301, 302 away from each other, so that the puncture guide 300 assumes the expanded position in absence of an external force.

In use, a surgeon can hold the puncture guide 300 at the arms 310, 311 and compress the arms 310, 311, so that the puncture guide 300 assumes the closed position. The surgeon can then introduce the guide plates 301, 302 into one nasal passage of the patient, and subsequently release pressure from the arms 310, 311, so that the guide plates 301, 302 spread apart from each other and spread the nasal passage of the patient. In such spread position, the surgeon can then perform the treatment method as described above for the first nasal passage of the patient. Afterwards, the surgeon can remove the puncture guide 300 from the first nasal passage and repeat the procedure for the second nasal passage.

In use, only one of the guide plates 301, 302 will abut the nasal wall of the patient, while the other one of the guide plates 301, 302 will abut the nasal septum. Only the puncture passages in the guide plate contacting the nasal wall will be used during treatment.

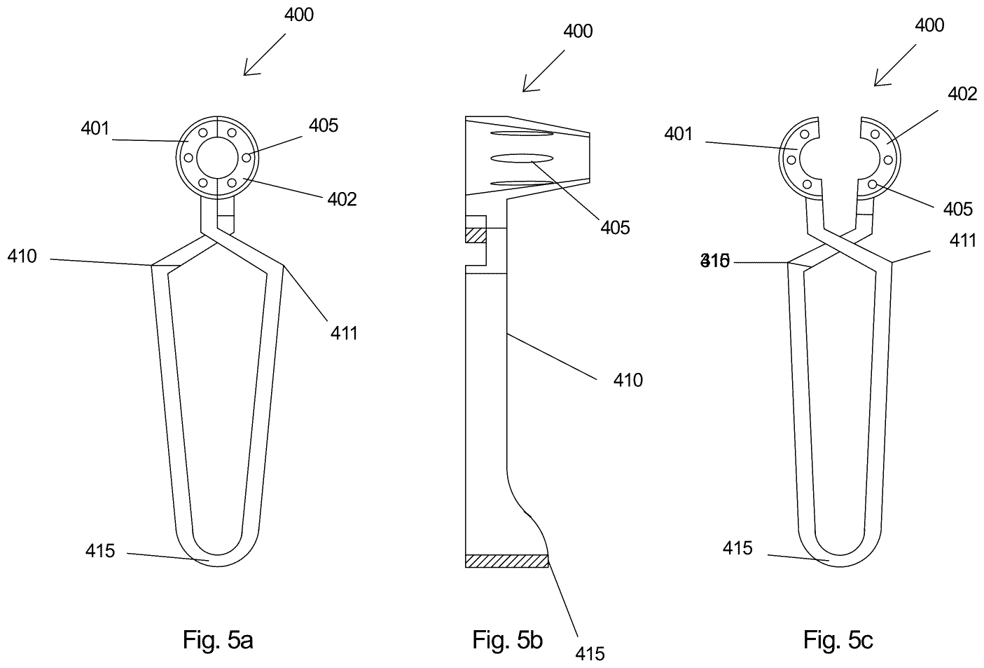

FIGS. 5a to 5c show a further embodiment of a puncture guide. The puncture guide 400 again comprises first and second guide plates 401, 402, each guide plate 401, 402 comprising three puncture passages 405. Arms 410, 411 attached to the guide plates 401, 402 are connected through an elastic joint 415 at their other ends. FIG. 4b shows puncture guide 400 in a sectional side view.

Other than the arms 310, 311 of puncture guide 300, the arms 410, 411 of puncture guide 400 are crossed between the guide plates 401, 402 and the elastic joint 415, so that the elastic joint 415 biases the puncture guide 400 into the closed position shown in FIG. 5a, and the puncture guide 400 can be brought into the expanded position shown in FIG. 5c by compressing the arms 410, 411.

In use, a surgeon can hold the puncture guide 400 at the arms 410, 411 without compressing the arms 410, 411, so that the puncture guide 400 is in the closed position, and introduce the guide plates 401, 402 into one nasal passage of the patient. The surgeon can then carefully push the arms 410, 411 towards each other so that the guide plates 401, 402 move away from each other and spread the nasal passage of the patient. In such spread position, the surgeon can then perform the treatment method as described above for the first nasal passage of the patient. Afterwards, the surgeon can remove the puncture guide 400 from the first nasal passage and repeat the procedure for the second nasal passage.

1. A method for treatment of nasal wall insufficiency, comprising:
a) providing a puncture guide, the puncture guide comprising a stabilizing frame and a plurality of puncture passages,
b) providing a bipolar radiofrequency probe having a piercing tip,
c) introducing the puncture guide into the nasal passages of the patient,
d) piercing the radiofrequency probe into nasal wall tissue through one of the plurality of puncture passages,
e) energizing the radiofrequency probe for heating the nasal wall tissue,
f) de-energizing the radiofrequency probe,
g) removing the radiofrequency probe from the nasal wall tissue,
h) repeating steps d) to g) for sequentially pierce the radiofrequency probe through each of the plurality of puncture passages,
i) allowing the nasal wall tissue to cool.

2. The method of claim 1, wherein the puncture guide is left in the nasal passages of the patient until the method is completed.

3. The method of claim 1, wherein the puncture guide is removed from the nasal passage of the patient about 2 minutes after de-energizing the radiofrequency probe for the last time.

4. The method of claim 1, wherein the nasal wall tissue conforms to a shape of the puncture guide during treatment.

5. The method of claim 4, wherein the nasal wall tissue fully or partially maintains the amended shape after cooling.

6. The method of claim 1, wherein the puncture guide is configured to be introduced into both nasal passages of the patient simultaneously.

7. The method of claim 1, wherein the puncture guide is configured to be introduced into the nasal passages of the patient one after the other.

8. A puncture guide for treatment of nasal wall insufficiency, comprising a stabilizing frame and a plurality of puncture passages.

9. The puncture guide of claim 8, wherein the frame of the puncture guide includes at least one hollow plug.

10. The puncture guide of claim 9, wherein the frame of the puncture guide includes a first hollow plug for introduction into the right nasal passage of a patient, and a second hollow plug for introduction into the left nasal passage of a patient.

11. The puncture guide of claim 10, wherein the frame of the puncture guide includes a tether between the first hollow plug and the second hollow plug.

12. The puncture guide of claim 10, wherein the first hollow plug comprises a first plurality of puncture passages, and the second hollow plug comprises a second plurality of puncture passages.

13. The puncture guide of claim 9, wherein the puncture guide includes a handle attached to the hollow plug.

14. The puncture guide of claim 8, wherein the puncture guide comprises:
a first guide plate and a first arm extending proximally from the first guide plate,
a second guide plate and a second arm extending proximally from the second guide plate, and
a plurality of puncture passages provided in the first guide plate.

15. The puncture guide of claim 14, wherein the puncture guide further comprises a plurality of puncture passages provided in the second guide plate.

16. The puncture guide of claim 14, wherein the first and second guide plates are curved.

17. The puncture guide of claim 14, wherein the first arm and the second arm merge in a proximal elastic joint.

18. An instrument set for treatment of nasal wall insufficiency, comprising:
a bipolar radiofrequency probe, the probe having a main body and an elongate shaft, the shaft having a piercing tip and first and second electrodes disposed near the distal end thereof, and
the puncture guide of claim 8.

19. A medical system for treatment of nasal wall insufficiency, the system comprising:
a bipolar radiofrequency probe, the probe having a main body and an elongate shaft, the shaft having a piercing tip and first and second electrodes disposed near the distal end thereof,
a puncture guide having a stabilizing frame and a plurality of puncture passages, and
an electrosurgical generator.
